(19) United States
(12) Patent Application Publication
(10) Pub. No.: US 2009/0143784 A1
(43) Pub. Date: Jun. 4, 2009

(54) Tibial Aiming Device For The Double Channel Technique

(76) Inventors: Wolf Petersen, Berlin (DE); Thore Zantop, Muenster (DE); Sascha Berberich, Tuttlingen (DE); Michael Sauer, Tuttlingen (DE); Sabine Summerer, Tuttlingen (DE)

Correspondence Address: ST. ONGE STEWARD JOHNSTON & REENS, LLC, 986 BEDFORD STREET, STAMFORD, CT, 06905-5619, US

(21) Appl. No.: 12/275,756

(22) Filed: Nov. 21, 2008

(30) Foreign Application Priority Data
Nov. 23, 2007 (DE) 10 2007 057 075.0

(51) Int. Cl. A61B 17/58

(52) U.S. Cl. 606/96; 606/88

(57) ABSTRACT

A device (10) serves to target and introduce bore channels into a tibia in the vicinity of a knee joint during reconstruction of the front cruciate ligament. It has a handle (12), a guide sleeve (18) removably attached to said handle, said guide sleeve having a longitudinal axis (44). A distal end of said guide sleeve (18) constitutes a first aiming point. An arm (28) protrudes from said handle (12). A distal end of said arm constitutes a second aiming point. A first opening (42) is provided in a distal end region of said arm, said opening (42) being in alignment with said longitudinal axis (44). A second opening (50) is provided in that distal end region of said arm, said second opening (50) is at a distance from said first opening (42).

### BACKGROUND OF THE INVENTION

The invention relates to a device for targeting and introducing bore channels into the tibia in the vicinity of the knee joint during reconstruction of a front cruciate ligament.

Such a tibial aiming device for the front cruciate ligament is known from the catalogue “Arthroscopy, Sports Medicine, Spinal Surgery, 2nd edition January/2005, page 71” by Karl Storz GmbH & Co. KG, Tuttlingen, Germany.

This device has a handle, with a guide sleeve attached removably on the handle, the distal end of which constitutes a first aiming point on the bone, with a protruding arm, whereof the distal end constitutes a second aiming point on the tibial plateau, whereby in the region of its distal end the arm has a first opening which is in alignment with a longitudinal axis of the guide sleeve such that a target wire guided through the guide sleeve encounters the first opening after penetrating the bone when the device is placed on the tibia.

The front cruciate ligament of the knee constitutes one of the two important ligaments which hold the knee joint. The second ligament is the rear cruciate ligament.

In particular, the front cruciate ligament is subject to very high stresses which can result in the front cruciate ligament tearing. The front cruciate ligament extends from the upper plateau (tibial plateau) of the lower leg bone (tibia) and runs to the inside of the lower end of the upper leg bone (femur).

During reconstruction of the front cruciate ligament this is replaced either by another natural tendon or by an artificial tendon implant.

For this purpose, a bore is made from the outside of the tibia, exiting at the level of the tibial plateau, specifically at the site where the natural cruciate ligament attaches. The alignment of this bore is such that it corresponds approximately to the natural alignment of the front cruciate ligament, that is to say the longitudinal extension, starting out from the tibial plateau, to the inside of the lateral femoral condyle.

The bore is then guided through the femur until it exits to the outside thereof.

The tendon implant or the substitute ligament is then inserted in both bore channels and fastened accordingly so that it can take on the function of the natural front cruciate ligament.

For successful reconstruction of the front cruciate ligament it is crucial that the bore channel made from the outside of the tibia is in an anatomical alignment which best approximates the alignment of the cruciate ligament, in a specific knee position.

During arthroscopy the surgeon has available a relatively restricted field of vision and also only relatively little space in the knee joint for manipulation.

The exit point of the bore channel introduced from the outside of the tibia, which lies on the tibial plateau, is thus barely seen, such that it is very difficult to target this point from the outside.

The abovementioned tibial aiming devices have accordingly come to prominence. The distal end region of the arm protruding from the device can be inserted between tibia and femur into the knee joint and its tip can be fixed at a point which corresponds approximately to the exit point of the tibial bore channel. In this distal end region of the arm there is an opening which aligns with the longitudinal axis of the guide sleeve.

The distal end of the guide sleeve is affixed to the bone from the outside, specifically aligned such that the longitudinal axis of the guide sleeve corresponds approximately to the orientation of the longitudinal extension of the front cruciate ligament.

If the aiming device is affixed in this way and placed properly a target wire is pushed in through the guide sleeve first, which, after it has penetrated the tibia, exits at the level of the tibial plateau from the bone and enters the opening on the distal end region in the vicinity of the tip.

The aiming device can now be removed and a hollow drill, which then bores out the bore channel in the tibia, can be pushed or guided via the target wire.

The target wire already driven into the tibia can be pushed in further to align and orient the bore channel in the femur, at a specific angled position of the knee, until it penetrates the femur such that the bore channel can then also be made in the femur identically.

From precise anatomical observation of the front cruciate ligament it is ascertained that, starting out from the tibial plateau, the front cruciate ligament splits into two slightly diverging bundles, specifically the so-called anteromedial bundle (AM) and the posterolateral bundle (PL).

This means that both starting points of these bundles are spaced slightly apart from one another on the femur.

In the case of the abovementioned operating technique this particular anatomical feature was not considered, but both AM and PL bundles were viewed as one common tendon strand and the corresponding bore channel was contrived such that it lies approximately in the middle of both bundles.

Since an aim in the reconstruction technique is to reconstruct the cruciate ligament as anatomically precisely as possible, an operating technique has been developed which takes into account the circumstance where the front cruciate ligament, when viewed from the tibial plateau, runs along both diverging bundles. If reconstruction is to be carried out here as closely as possible to anatomical conditions, it is sensible in reconstruction to use two tendons or transplants which extend on the one hand along the longitudinal axis of the anteromedial bundle and on the other hand along the longitudinal axis of the posterolateral bundle. Yet it is crucial here that the required two bore channels can be set in an alignment that is a divergence, which most closely approaches the longitudinal extension of the anteromedial or the posterolateral bundle. This technique is also called the double channel technique.

It is therefore an object of the present invention to further develop an aiming device of the abovementioned type such that with one and the same aiming device two bore channels can be targeted and introduced, which run in their orientation as closely as possible to the longitudinal extension of the anteromedial and posterolateral bundle.

### SUMMARY OF THE INVENTION

The object is solved by a device for targeting and introducing bore channels into a tibia in a vicinity of a knee joint during reconstruction of a front cruciate ligament, comprising a handle, a guide sleeve removably attached to said handle, said guide sleeve having a longitudinal axis, a distal end of said guide sleeve constitutes a first aiming point on an outside of a tibia bone, an arm protruding from said handle, a distal end of said arm constitutes a second aiming point on a tibial plateau, a first opening being provided in a distal end region of said arm, said opening being in alignment with said longitudinal axis of said guide sleeve, a target wire, guided through said guide sleeve can enter said first opening, and a second opening being provided in said distal end region of said arm, said second opening is at a distance from said first opening, said distance corresponds to a distance between an anteromedial bundle and a posterolateral bundle of a front cruciate ligament projecting from a tibial plateau of a tibia.

Providing this second opening now enables two target wires to be set successively using one and the same aiming device, the alignment or orientation of which corresponds to the orientation of the anteromedial or the posterolateral bundle.

The procedure here is that a first tibial bore channel is first created which corresponds for example to the orientation and extension of the anteromedial bundle. For this purpose, the aiming device is accordingly affixed via both its aiming points, i.e., the distal end of the guide sleeve on the outside of the tibia and the tip on the tibial plateau, then the first target wire is driven through. This target wire then has the alignment of the longitudinal extension of the anteromedial bundle. From here on the guide sleeve is removed or pulled off from the first target wire; at the same time the end of the first target wire protruding from the tibial plateau is still located in the first opening. The arm is now moved in such a way that this protruding end exits from the first opening and this protruding end is now pushed into the second opening. The guide sleeve is again inserted in the device and the aiming device is reattached to the tibia, specifically such that from here on the longitudinal axis of the guide sleeve extends in the direction of the posterolateral bundle.

By selecting the distance between the first opening and the second opening, the position and divergence corresponding to the position and divergence of both bundles of the front cruciate ligament can be preset. From here on a second target wire is pushed through the guide sleeve and the bone until it likewise exits on the tibial plateau, though slightly offset medially to the already set first target wire. Since the middle longitudinal axis of the guide sleeve is in alignment with the first opening, the procedure ensures that both target wires do not hinder or contact one another. This secure guiding, the orientation and alignment are also guaranteed by the first target wire being housed in the second opening when the second target wire is set, thus having a preset orientation to the second target wire.

In this way, using one and the same aiming device it is possible to set two bore channels or target wires via a relatively simple procedure, which extend according to the anatomical alignment both of the anteromedial and of the posterolateral bundle. After the second target wire is set the guide sleeve is then pulled off this wire and the device can be removed from the knee. Both target wires are now sticking in the tibia and the boring procedures and also the introduction of the tibial bore channels can then be performed.

In a further configuration of the invention the second opening is at a distance proximally from the first opening.

The advantage of this step is that with respective targeting of the second aiming point, that is to say the tip of the arm, the view of this tip is guaranteed, also whenever the previously set first target wire is housed in the second opening, as this protruding tip is visible from almost all positions.

In a further configuration of the invention the second opening is designed as an elongated hole opening.

The advantage of this step is that when the second target wire is being set the distance from the middle longitudinal axis of the first target wire can be varied slightly, depending on whether the set first target wire is located at one or the other end of the elongated hole. If the set first target wire is located at the end of the elongated hole which lies in the vicinity of the first opening, the offset or the distance of the openings is less than if the set first target wire were located at the opposite end of the elongated hole, which would be further removed from the first opening.

These end positions are very easily reached in that the surgeon either draws the aiming device slightly towards himself or pushes it away such that the first set target wire is located at either one or the other end of the elongated hole opening. An added advantage here is that unwanted or error positions are not created which go beyond the anatomical variation possibilities.

In a further configuration of the invention the first opening is designed as an elongated hole.

The advantage of this step is that the configuration as elongated hole opening makes it easier to remove the arm from the first target wire by its first end driven through the bone, after the first target wire has been set. This can take place for example by lateral tipping of the device such that when the end projects only a few millimetres beyond the tibial plateau, this is sufficient for this end to exit from the elongated hole opening.

In a further configuration of the invention the first opening is open to the side such that a first target wire guided through said guide sleeve and housed in the first opening can be moved sideways out of the opening.

The advantage of this configuration is that the target wire housed in the first opening can exit from the latter through lateral shifting of the distal end region of the arm. This is also possible for example if the first target wire has been driven in considerably far beyond the tibial plateau, with exiting possible via the lateral opening, independently of how far this end projects.

In a further configuration of the invention the distance of the midpoints of first and second opening is ca. 8-10 mm.

It has eventuated that this distance measurement takes into account the anatomical conditions of a person to achieve the corresponding orientation of anteromedial and posterolateral bundle.

In a further configuration of the invention the openings are designed as bores penetrating through the body of the arm.

The advantage of these steps is not only that these openings are easy to make, but also allow the ends of the target wires exiting from the tibial plateau to be pushed fully through the body of the arm. It would also be adequate in theory to form the openings merely as troughs or notches in the body, although it would then have to be ensured that the ends exiting on the tibial plateau exit only precisely so far that they fit into these troughs.

Handling therefore becomes easier still by the openings being designed as continuous openings. In particular, in connection with the abovementioned step, according to which the first opening is still open to the side, handling is made considerably easier.

It is understood that the abovementioned characteristics and those yet to be explained hereinbelow can be used not only in the specified combinations, but also in other combinations or alone, without departing from the scope of the present invention.

### BRIEF DESCRIPTION OF THE DRAWINGS

The invention will now be described and explained in greater detail by means of a selected exemplary embodiment in conjunction with the attached diagrams, in which:

FIG. 1 is a perspective view of a device, of the invention,

FIG. 1a is an enlarged illustration of the region bounded in FIG. 1 by a circle,

FIG. 2 highly schematically illustrates a human leg in the vicinity of the knee joint to elucidate the orientation of the anteromedial and of the posterolateral bundle of the front cruciate ligament,

FIG. 3 shows the use of the device of FIG. 1 when a first tibial target wire is set, whereby the femur is not illustrated for the sake of clarity,

FIG. 4 is an illustration corresponding to FIG. 3 after the first target wire is set,

FIG. 5 shows a situation after the guide sleeve has been removed from the first, already set, target wire and whereof the end protruding beyond the tibial plateau was moved out of the first opening and threaded into the second opening,

FIG. 6 shows a situation in which a second target wire was set, and

FIG. 7 shows the tibia with both set target wires after the device has been removed.

### DETAILED DESCRIPTION OF A PREFERRED EMBODIMENT

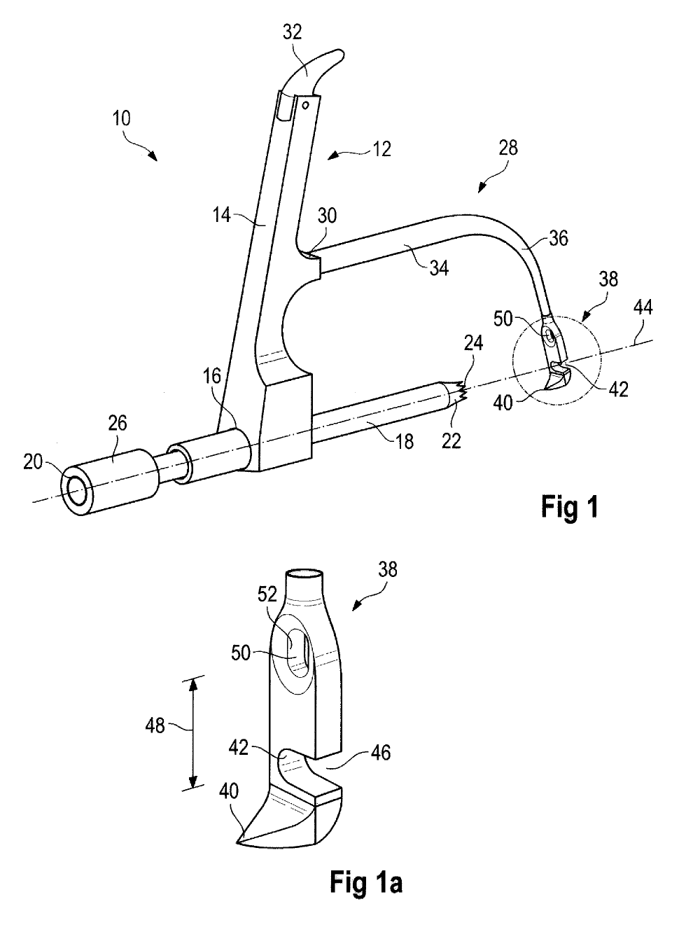

A device illustrated in FIG. 1 or 1a is designated in its entirety by reference numeral 10.

The device 10 has a handle 12 which has a straight, long stretched-out rod-like grip 14. A guide sleeve 18 is inserted in an opening 16 at an end region. The guide sleeve 18 has a tube 20 whereof the distal end 22 is fitted with a notched crown 24. At the opposite end the guide sleeve 18 is provided with a clamping sleeve 26. The clamping mechanism is designed such that turning the clamping sleeve 26 in one direction produces localized retaining of the guide sleeve 18, for example in the alignment or shift position illustrated in FIG. 1.

If the clamping sleeve 26 is rotated in the opposite direction the guide sleeve 18 can be moved reciprocally along its longitudinal axis 44, or can also be completely removed from the grip 14, to the left in the illustration of FIG. 1.

An arm 28 protrudes from the rod-like grip 14, viewed approximately at half height. The arm 28 is housed in a mounting 30, and can be fixed by a clamping lever 32 or correspondingly loosened, i.e. removed.

The arm 28 has a straight section 34 extending approximately parallel to the guide sleeve 18. The straight section 34 merges via a curved section 36 into a distal end region 38. At the outer distal end a tip 40 is configured which faces the notched crown 24 of the guide sleeve 18.

As is evident in particular from the enlarged illustration of FIG. 1a, a first opening 42 is recessed in the distal end region 38 in the body of the arm 28. The first opening 42 is designed as an opening fully penetrating the body of the arm 28. The first opening 42 is open to the side via a groove 46.

The middle longitudinal axis of the first opening 42 aligns with the longitudinal axis 44 of the guide sleeve 18.

This means that if for example a target wire, as hereinbelow yet to be described, is pushed in through the guide sleeve 18 from the proximal side, in the illustration of FIG. 1 from the left side, it precisely meets the first opening 42.

At a distance 48 from the middle longitudinal axis of the first opening 42 a second opening 50 is provided in the distal end region 38, the orientation of which is identical to that of the first opening 42.

As is evident in particular from FIG. 1a, the second opening 50 is designed as an elongated hole 52, whereby the longer axis extends distally to proximally.

It is likewise evident that the second opening 50, viewed from the tip 40, is at a distance 48 proximally from the first opening 42. As is evident in particular from FIG. 1a, the second opening 50 is strongly chamfered.

The distance 48 is approximately 8 to 10 mm.

The length of the longer axis of the elongated hole 52 is approximately 8 to 10 mm.

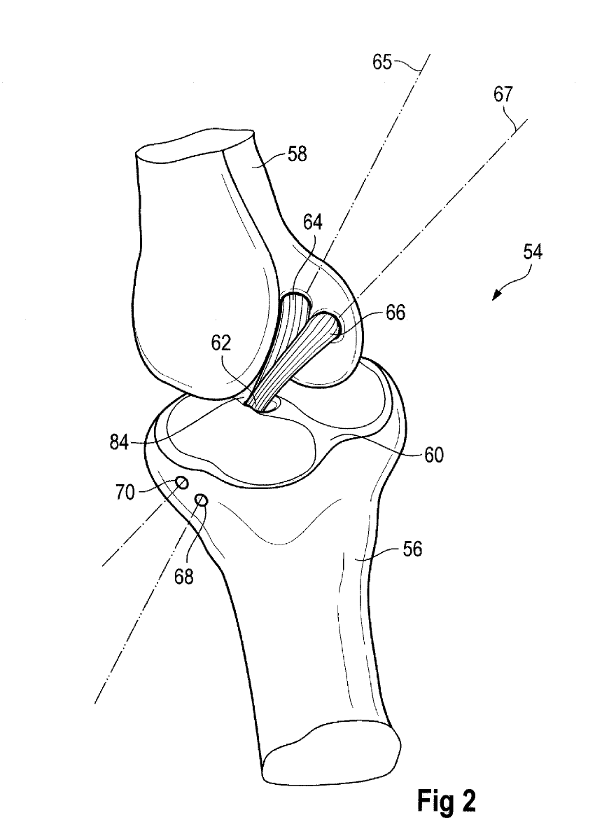

The sense and purpose of this configuration will be described and explained in greater detail hereinbelow by way of the sequence of figures from FIG. 2 to FIG. 7.

FIG. 2 schematically illustrates a section of a human leg 54 in the vicinity of the knee joint, whereby the tibia 56, that is to say the lower leg bone, and the femur 58, that is to say the upper leg bone, are evident. From the tibial plateau 60 of the tibia 56 the front cruciate ligament 62 extends, specifically dorsally as far as the inside of the lateral femoral condyle.

It is evident that the front cruciate ligament 62, starting out from the tibial plateau 60, has an anteromedial bundle 64 and a posterolateral bundle 66.

The corresponding longitudinal axes 65 and 67 diverge, when viewed starting out from the tibial plateau 60.

The sense and purpose of the device 10 from here on is to make bore channels 68 and 70 in the tibia 56, whereof the orientation corresponds to the longitudinal axes 65 or 67 of the anteromedial or of the posterolateral bundle.

If both these longitudinal axes 65 and 67 are traced as far as the tibial plateau 60, it is obvious that they are at a slight medial distance from one another and also diverge slightly. The bore channels 68 and 70 accordingly must be oriented and contrived such that they correspond to this anatomical alignment.

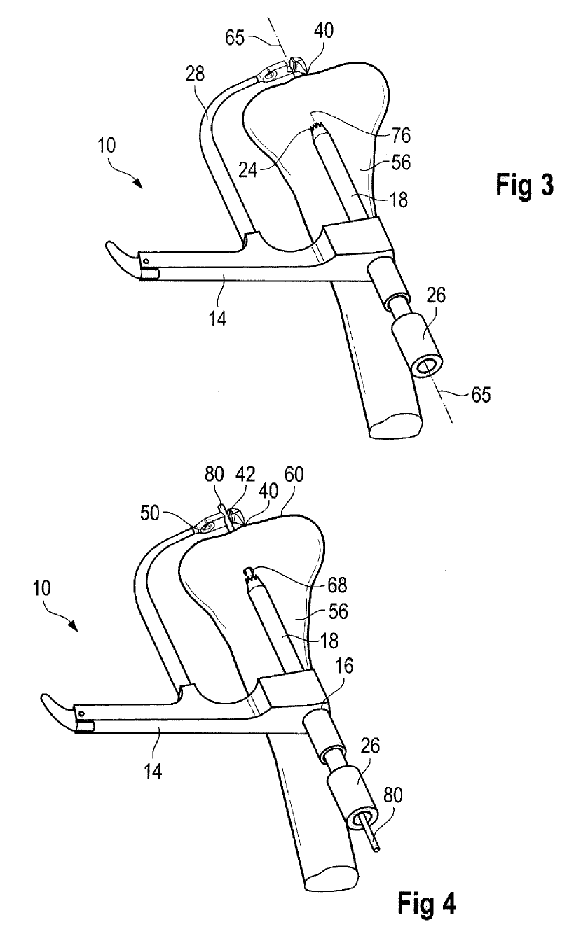

With reference to FIG. 3, for this purpose the device 10 is placed on the tibia 56, in the manner evident therefrom. The purpose of the notched crown 54 is to define a first aiming point 76 on the outside of the bone in a firmly sitting manner, which lies just under the widening of the tibia. The distal end of the arm 28 was fed through a lateral opening of the knee between tibia 56 and femur 58 into the knee joint socket. The tip 40 constitutes a second aiming point 78. The surgeon can observe this orientation during an arthroscopic procedure, using an endoscope for example. When the clamping sleeve 26 is rotated the device 10 is held in a firm and secure position, supported still by the notched crown 24 and the tip 40 penetrating slightly into the bone.

As is evident from FIG. 4, from here on a first target wire 80 is pushed proximally to distally through the guide sleeve 18, penetrates the bone, and thus offers the exit point for a first bore channel 68. The first target wire 80 is advanced to the point where it exits again at the level of the tibial plateau 60 and at the same time enters the first opening 42. The longitudinal axis of the set first target wire 80 extends along the longitudinal axis 65 of the anteromedial bundle 64, as pointed out in FIG. 2.

The clamping sleeve 26 is now twisted and the guide sleeve 18 can be pulled off both the handle 12 and the set first target wire 80.

Next, the distal end region 38 of the arm 28 is moved such that the end of the first target wire 80 protruding beyond the tibial plateau 60 exits sideways from the first opening 42. This protruding end is then introduced into the second opening 50, after which the guide sleeve 18 is reinserted in the handle 12.

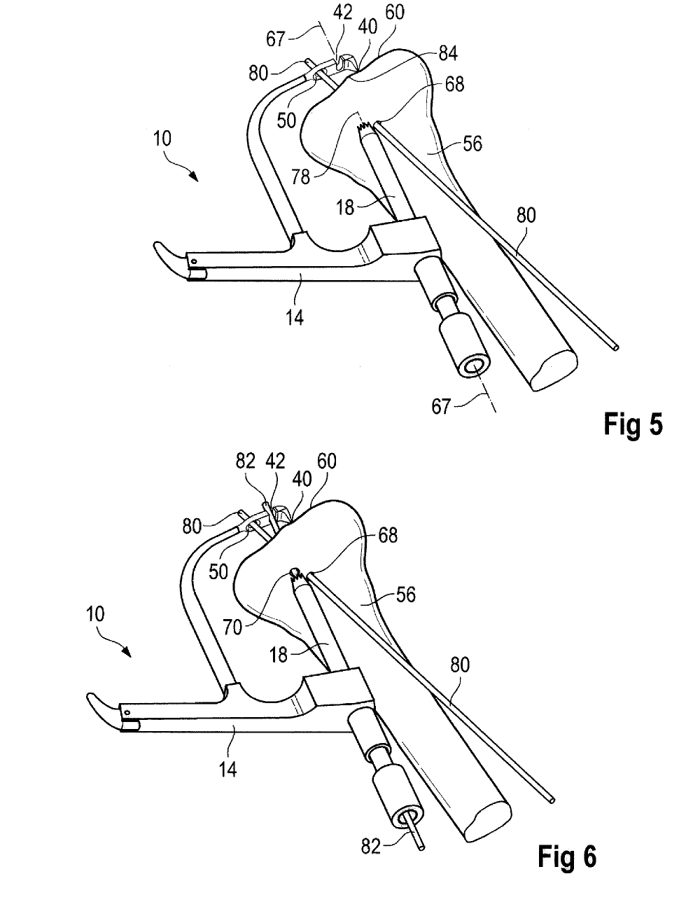

This situation is illustrated in FIG. 5. It is thus evident here that the end of the first target wire 80 protruding beyond the tibial plateau 60 is inserted into the second opening 50, and that the first target wire 80 is no longer housed in the guide sleeve 18.

The surgeon now orients the device 10 such that the guide sleeve 18 or the opening 42 is aligned for the latter to extend in the longitudinal extension of the longitudinal axis 67 of the posterolateral bundle 66. At the same time, the exit point out of the tibial plateau 60 is selected such that this exit is offset medially along the intercondylar region 84. This orientation is made all the easier by the first target wire 80 being housed in the second opening 50. As mentioned earlier, the second opening 50 is designed as an elongated hole. If the surgeon pulls the rod-like grip 14 slightly proximally, therefore towards himself, the first target wire 80 moves in the elongated hole opening 52 such that it is placed at the end of the elongated hole which is closer to the opening 42.

On the contrary, if he pushes the grip 14 away from himself the first target wire 80 moves in the direction of the opposite end of the elongated hole, that is to say at the end farthest from the first opening 42.

This configuration allows him to thus slightly vary the distance so as to be able to adapt to anatomical conditions.

If the device 10 or the longitudinal axis 44 of the guide sleeve 18 is in the corresponding desired alignment, that is to say in the alignment of the longitudinal axis 67 of the posterolateral bundle, a second target wire 82 is set, as is evident in FIG. 6.

The second target wire 82 enters the bone and thus defines the entry point for a second bore channel 70. This second target wire 82 is guided in a defined manner into the first opening 42 in such a way as to exclude both these target wires 80, 82 making contact or hindering each other.

Next, the guide sleeve 18 is stripped away from the second target wire 82, and the distal end region 38 of the arm 28 is then moved such that the end of the second target wire 82 projecting beyond the tibial plateau 60 is moved out of the laterally open first opening 42, then the distal end region 38 is pulled off over the correspondingly protruding end of the first target wire 80 and the device 10 can be removed from the operation site.

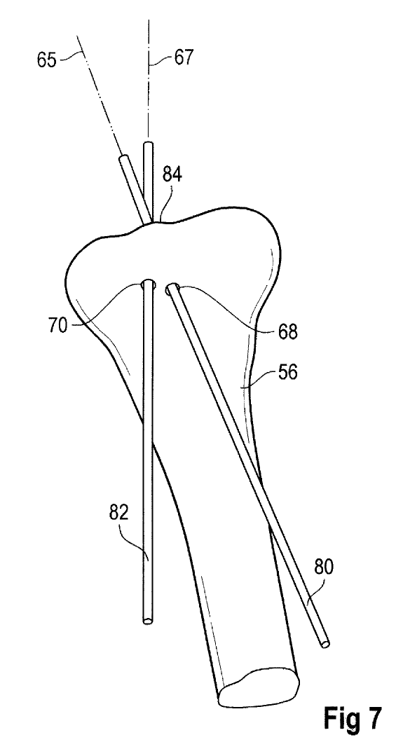

This situation is illustrated in FIG. 7.

From here on two target wires 80 and 82 are set by means of the inventive device 10 oriented anatomically correctly, the longitudinal axes of which on the one hand extend in the longitudinal axis 65 of the anteromedial bundle and on the other hand extend in the longitudinal axis 67 of the posterolateral bundle.

A hollow drill can now be brought over the target wires 80 and 82 and the corresponding bore channels can be made in the tibia. The target wires 80 and 82 can also be pushed further forwards to be driven through the femur as far as its outside, for example in this orientation, whereby this then occurs in an angled-off position of the knee, which favours reconstruction. Here, too, the channels can then be made in the femur by overthrusting corresponding hollow drills.

Two skeins of a tendon replacement are then correspondingly pushed into the resulting bore channels 68 and 70 and fastened, which can be done by way of techniques known per se. Accordingly, both these skeins are guided in through the bore channels in the femur and fixed there, as is likewise known per se. As a result, both tendon replacement skeins then extend along the longitudinal axes of the anteromedial or posterolateral bundle such that reconstruction most closely approximating the anatomy of the cruciate ligament can be carried out.

The previously described orientation of the device 10 and target wires 80 and 82 explained in the sequence of FIGS. 3 to 7 served to explain the application in principle of the device on the tibia 56. For illustrative reasons the femur 58 was omitted and the anatomically exact tibial exit point of the front cruciate ligament was not taken into account. Crucial here is the bone bridge between the tibial exit points of both target wires 80 or 82, i.e. the later bore channels, the trajectory of both bore channel (or target wire) axes along the anteromedial (AM) and the posterolateral (PM) bundle, and the position of the exit points from the tibial plateau along the intercondylar region 84.

What is claimed is:

1. A device for targeting and introducing bore channels into the tibia in a vicinity of a knee joint during reconstruction of a front cruciate ligament, comprising
a handle,
a guide sleeve removably attached to said handle, said guide sleeve having a longitudinal axis,
a distal end of said guide sleeve constitutes a first aiming point on an outside of a tibial bone,
an arm protruding from said handle,
a distal end of said arm constitutes a second aiming point on a tibial plateau,
a first opening being provided in a distal end region of said arm, said first opening being in alignment with said longitudinal axis of said guide sleeve, wherein
a second opening being provided in said distal end region of said arm, said second opening is at a distance from said first opening, said distance corresponding to a distance between an anteromedial bundle and a posterolateral bundle of a front crucial ligament projecting from a tibial plateau of a tibia.

2. The device of claim 1, wherein said second opening is at said distance proximally from said first opening.

3. The device of claim 1, wherein said the second opening is designed as an elongated hole opening.

4. The device of claim 1, wherein said first opening is designed as an elongated hole.

5. The device of claim 1, wherein said first opening is open to a side, such that a target wire guided through said guide sleeve and housed in said first opening can be moved sideways out of said first opening.

6. The device of claim 1, wherein said distance between midpoints of said first and said second opening is in a range of about 8 to about 10 mm.

7. The device of claim 1, wherein said first and said second openings are designed as bores penetrating through a body of said arm.
